(19) United States
(12) Patent Application Publication
(10) Pub. No.: US 2018/0220608 A1
(43) Pub. Date: Aug. 9, 2018

(54) A Method for Increasing the Content of Notoginsenoside R1 in Panax Notoginseng

(71) Applicant: Guangxi Botanical Garden of Medicinal Plant, Nanning, Guangxi (CN)

(72) Inventors: Qianping Chen, Nanning, Guangxi (CN); Longhua Bai, Nanning, Guangxi (CN); Changming Mo, Nanning, Guangxi (CN); Limei Pan, Nanning, Guangxi (CN); Hao Huang, Nanning, Guangxi (CN); Rongchang Wei, Nanning, Guangxi (CN); Shixin Feng, Nanning, Guangxi (CN); Ni Jiang, Nanning, Guangxi (CN); Yunfeng Ye, Nanning, Guangxi (CN)

(73) Assignee: Guangxi Botanical Garden of Medicinal Plant, Nanning, Guangxi (CN)

(21) Appl. No.: 15/749,677

(22) PCT Filed: Jul. 28, 2016
(86) PCT No.: PCT/CN2016/092052

§ 371 Date: Feb. 1, 2018

(30) Foreign Application Priority Data
Aug. 5, 2015 (CN) 201510472498.6

(51) Int. Cl. A01H 3/04; A01H 6/00; A01C 21/00; A01G 2/00

(52) U.S. Cl. CPC A01H 3/04; A01G 2/00; A01C 21/005; A01H 6/00

(57) ABSTRACT

The invention discloses a method for improving the content of notoginsenoside R1 in Panax notoginseng, in which the Panax notoginseng plants are sprayed with salicylic acid solution before the Panax notoginseng s are harvested. The method of the present invention can improve the content of notoginsenoside R1 in Panax notoginseng by the pretreatment of Panax notoginseng plants with the salicylic acid solution before harvesting, and can be used in large scale in the conventional cultivation method of Panax notoginseng so as to facilitate large-scale industrialization Production of notoginsenoside R1, to meet the market demand for notoginsenoside R1.

### TECHNICAL FIELD

The present invention relates to the field of Panax notoginseng cultivation, more particularly, the present invention relates to a method for increasing the content of notoginsenoside R1 in Panax notoginseng.

### BACKGROUND TECHNIQUE

Panax notoginseng is the dry root of Araliaceae Plant Panax notoginseng (Burk.) FHchen dry roots, and is the traditional precious medicinal herbs, with hemostasis, blood stasis dissipating, detumescence, pain relieving and more other effects, and the preparations prepared with the Panax notoginseng or total saponins in the clinical are widely used in the treatment of cardiovascular and cerebrovascular diseases. Notoginseng saponin R1 belongs to the original ginsenetriol saponins, is the unique chemical constituents in Panax notoginseng, also is the most representative characteristic compounds, and the previous study has proved that notoginsenoside R1 has good curative effect to prevent vascular endothelial cell injury. Notoginsenoside R1 belongs to the original ginsenetriol saponins type, is the most representative characteristic compound in the Panax notoginseng, has a wide range of pharmacological effects, has been used as the main component of coronary heart Danshen Dripping Pill for the treatment of myocardial ischemic disease. Anti-inflammation is one of the important pharmacological effects of notoginsenoside R1. Previous studies have proved that notoginsenoside R1 has good curative effect on vascular endothelial cell injury. Due to the long production cycle of Panax notoginseng and the low content of notoginsenoside R1 in Panax notoginseng, and the chemical synthesis method is difficult to be scaled to industrial production, therefore, it is particularly important whether to find a way to improve the content of notoginsenoside R1. So far, there is no report on increasing the content of notoginsenoside R1 in Panax notoginseng cultivation.

### SUMMARY OF THE INVENTION

It is an object of the present invention to solve at least the above problems and to provide at least the advantages which will be described later.

It is a further object of the present invention to provide a method for increasing the content of notoginsenoside R1 in Panax notoginseng, and to solve the problem that the content of notoginsenoside R1 in Panax notoginseng is too low.

It is also an object of the present invention to increase the content of notoginsenoside R1 in Panax notoginseng by spraying the salicylic acid solution to the leaves of Panax notoginseng plants before the Panax notoginseng harvest, and can be used in large scale in the conventional cultivation method of Panax notoginseng so as to facilitate large-scale industrialization Production of notoginsenoside R1, to meet the market demand for notoginsenoside R1.

In order to achieve these objects and other advantages according to the present invention, there is provided a method for increasing the content of notoginsenoside R1 in Panax notoginseng, in which Panax notoginseng plants are sprayed with salicylic acid solution before harvesting Panax notoginseng.

It is preferred that the method for increasing the content of notoginsenoside R1 in Panax notoginseng, wherein the salicylic acid solution has a concentration of 0.3-1.3 mmol/L.

It is preferred that the method for increasing the content of notoginsenoside R1 in Panax notoginseng, wherein the Panax notoginseng plants are sprayed with the salicylic acid solution on 30 to 70 days before the Panax notoginseng harvest.

It is preferred that the method for increasing the content of notoginsenoside R1 in Panax notoginseng, wherein the Panax notoginseng plants are those which have been cultivated for 2 to 3 years.

It is preferred that the method for increasing the content of notoginsenoside R1 in Panax notoginseng, wherein the salicylic acid solution is sprayed on the leaves surface of the Panax notoginseng plants.

It is preferred that the method for increasing the content of notoginseng saponin R1 in Panax notoginseng, wherein the Panax notoginseng plants are sprayed with the salicylic acid solution once every 10-20 days before harvest, totally 2-3 times.

It is preferred that the method for increasing the content of the notoginseng saponin R1 in Panax notoginseng, wherein the Panax notoginseng seeds are sowed in January of the first year, and the Panax notoginseng seedlings are obtained in January of the second year, which are planted after being transplanting to obtain planting seedlings. And in April to May planting seedlings are cut along the circumference close to the root, and the cuts are firstly sprayed with a chitosan aqueous solution with a mass fraction of 1.2%-1.5%, then coated with a plant mashing liquid, and then wrapped around with sponge with a thickness of 3-5 mm, wherein the sponge is immersed in the first soaking liquid for 20-22 h at a soaking temperature of 33-35° C. after sterilization.

The chitosan in the chitosan aqueous solution is a chitosan with a N-deacetyl degree of 85%-88%;

The plant mashing liquid is prepared by mixing aloe, moss and pepper in a mass ratio of 3:7:1, then mashing and grinding;

The first soaking liquid is made by mixing bamboo vinegar liquid, fatty alcohol polyoxyethylene ether sodium sulfate, peppermint essential oil and water in a mass ratio of 2-3:5-7:1-2:1000.

It is preferred that the method for increasing the content of the notoginsenoside R1 in Panax notoginseng is carried out 30 days before the Panax notoginseng harvest, and the salicylic acid solution is sprayed on the leaves of the Panax notoginseng plant at an interval of 10 days, totally 3 times, and non-woven sleeves are used for covering leaves of Panax notoginseng plants, then removed after 2-4 h, wherein the non-woven sleeves are immersed in the second soaking solution at 28-30° C. for 20-22 h, and the second soaking liquor is prepared by mixing sodium salicylate, salicylic acid and water at a mass ratio of 4-5:7-8:1000.

It is preferred that the method for increasing the content of the notoginsenoside R1 in Panax notoginseng, wherein in March of the third year, a plurality of pores is drilled on the main stem of the Panax notoginseng plant along the length direction of the main stem, and then sulfonic acid aqueous solution is sprayed with a sprayer on the pores, wherein the sulfonic acid aqueous solution is prepared by mixing sulfonic acid, sodium cocoyl isethionate and water in a mass ratio of 7:3-5:10000.

It is preferred that the method for increasing the content of the notoginsenoside R1 in Panax notoginseng, wherein before the sowing, the Panax notoginseng seeds are sterilized, then 3-5 holes are pinned on the surface of the Panax notoginseng seed. The seeds are further immersed in a third soaking liquid at 35° C. for 5-7 hours, coated with a film for 3-5 minutes and then removing the film, wherein the third soaking liquid is prepared by mixing salicylic acid, water-soluble chitosan, lanolin, ethanol and water in a mass ratio of 1:7-9:5:200:800.

The method for improving the content of notoginsenoside R1 in Panax notoginseng of the invention has the advantages of simple operation and low cost, and is applicable to the method of large-scale conventional planting of Panax notoginseng, and requires less to the personnel who plant Panax notoginseng. And at least the following beneficial effects are included:

1) The present invention sprays the salicylic acid solution on the leaves of Panax notoginseng before harvest to stimulate and induce the expression of the functional gene of notoginsenoside R1 biosynthesis, namely the glucosyltransferase gene, breaking the rate-limiting step in the synthesis process of notoginsenoside R1, resulting in accumulation of a large amount of notoginsenoside R1 in a short period, thereby increasing its content;

2) The present invention accelerates the synthesis of the notoginsenoside R1 by stimulating the plantlets of Panax notoginseng and inducing the biosynthesis functional gene expression of the notoginsenoside R1 from the wounds which are cut near the roots of the planting seedlings and sprayed with a chitosan aqueous solution. Plant mashed liquid on the one hand is to further stimulate the Panax notoginseng plants to synthesize notoginsenoside R1 with the use of pepper, on the other hand indirectly increases the content of notoginsenoside R1 through the use of aloe and moss to moisturize, sterilize, and promote the Panax notoginseng cell regeneration. In addition, the covering of the sponge soaked with the first soaking solution around the wounds can further stimulate the notoginsenoside R1 synthesis, and prolong the time of Panax notoginseng plants to synthesize notoginsenoside R1, in order to improve its content, wherein the bamboo vinegar solution can not only promote the panax notoginseng cells to absorb other ingredients sprayed, coated or wrapped in the wounds, but also promote the growth of Panax notoginseng; the fatty alcohol polyoxyethylene ether sodium sulfate can stimulate the cell injury at the wounds to expand; the essential oil can promote and prolong the biosynthesis of notoginsenoside R1.

3) Before harvesting, the present invention spray salicylic acid solution on the leaves of the Panax notoginseng plants, and cover the leaves with the non-woven sleeves soaked with the second soaking solution to increase the absorbance of the second soaking solution components in the non-woven sleeves during the leaf breathing process, thus prolonging the time of stimulating the notoginsenoside R1 biosynthesis and increasing the accumulation of notoginsenoside content.

4) Panax notoginseng plants can be harvested 2-3 years after planting. Even though it will take a longer time to harvest in the third years, the Panax notoginseng saponin content slightly increased. The inventors of the present invention also found that in March of the third year, the spray of sulfonic acid aqueous solution on the stem of Panax notoginseng plant after drilling small holes can further promote the synthesis and accumulation of Panax notoginseng saponins in stem.

5) Generally, Panax notoginseng seeds are being seedling for large-scale cultivation, before sowing, the Panax notoginseng seeds are pinned and then soaked in the third soaking solution, and coated with a film. The expression of panax notoginsenoside R1 biosynthesis functional gene could be further stimulated by salicylic acid and water-soluble chitosan in order to increase the content of notoginsenoside R1 in Panax notoginseng plants. And Lanolin and ethanol can promote the absorption of salicylic acid and water soluble chitosan by the seeds and receive their stimulations.

Other advantages, objects, and features of the present invention will be set forth in part in the description which follows, and in part will be understood by those skilled in the art from the study and practice of the invention.

### DETAILED DESCRIPTION

The invention will now be described in further details with reference to specific embodiments in order to enable those skilled in the art to refer to the specification in light of the description.

It is to be understood that the terms “have”, “having”, “include”, “including”, “comprise” and “comprising” as used herein do not exclude the presence or addition of one or more other elements or combinations thereof.

### EXAMPLE 1

A method for increasing the content of notoginsenoside R1 in Panax notoginseng, wherein the Panax notoginseng plants are sprayed with salicylic acid solution before Panax notoginseng harvest.

Wherein the method for increasing the content of notoginsenoside R1 in Panax notoginseng, the salicylic acid solution has a concentration of 0.3-1.3 mmol/L.

Wherein the method for increasing the content of notoginsenoside R1 in Panax notoginseng, the Panax notoginseng plants are sprayed with the salicylic acid solution 30 to 70 days before Panax notoginseng harvest.

Wherein the method for increasing the content of the notoginsenoside R1 in Panax notoginseng, the Panax notoginseng plants have been cultivated for 2 to 3 years.

Wherein the method for increasing the content of the notoginsenoside R1 in Panax notoginseng, the salicylic acid solution is sprayed on the leaves surface of the Panax notoginseng plants.

Wherein the method for increasing the content of notoginsenoside R1 in Panax notoginseng, the Panax notoginseng is sprayed with the salicylic acid solution once every 10-20 days before harvest, totally 2-3 times.

Wherein the method for increasing the content of the notoginseng saponin R1 in Panax notoginseng, the Panax notoginseng seeds are sowed in January of the first year, and the Panax notoginseng seedlings are obtained in January of the second year, which are planted after being transplanting to obtain planting seedlings. And in April to May planting seedlings are cut along the circumference close to the root, and the cuts are firstly sprayed with a chitosan aqueous solution with a mass fraction of 1.2% -1.5%, then coated with plant mashing liquid, and then wrapped around with sponge with a thickness of 3-5 mm, wherein the sponge is immersed in the first soaking liquid for 20-22 h at a soaking temperature of 33-35° C. after sterilization. Wherein

The chitosan in the chitosan aqueous solution is a chitosan with a N-deacetyl degree of 85% -88%;

The plant mashing liquid is prepared by mixing aloe, moss and pepper in a mass ratio of 3:7:1, then mashing and grinding;

The first soaking liquid is made by mixing bamboo vinegar liquid, fatty alcohol polyoxyethylene ether sodium sulfate, peppermint essential oil and water in a mass ratio of 2-3:5-7:1-2:1000.

Wherein the method for increasing the content of the notoginsenoside R1 in Panax notoginseng is carried out 30 days before the Panax notoginseng harvest, and the salicylic acid solution is sprayed on the leaves of the Panax notoginseng plant at the interval of 10 days, 3 times, and non-woven sleeves are used for covering leaves of Panax notoginseng plant, then removed after 2-4 h, wherein the non-woven sleeves are immersed in the second soaking solution at 28-30° C. for 20-22 h, and the second soaking liquor is prepared by mixing sodium salicylate, salicylic acid and water at a mass ratio of 4-5:7-8:1000.

Wherein the method for increasing the content of the notoginsenoside R1 in Panax notoginseng, in March of the third year, a plurality of pores is drilled on the main stem of the Panax notoginseng plant along the length direction of the main stem, and then sulfonic acid aqueous solution is sprayed with a sprayer on the pores, wherein the sulfonic acid aqueous solution is prepared by mixing sulfonic acid, sodium cocoyl isethionate and water in a mass ratio of 7:3-5:10000.

Wherein the method for increasing the content of the notoginsenoside R1 in Panax notoginseng, before the sowing, the Panax notoginseng seeds are sterilized, then 3-5 holes are pinned on the surface of the Panax notoginseng seed. The seeds are further immersed in a third soaking liquid at 35° C. for 5-7 hours, coated with a film for 3-5 minutes and then removing the film, wherein the third soaking liquid is prepared by mixing salicylic acid, water-soluble chitosan, lanolin, ethanol and water in a mass ratio of 1:7-9:5:200:800.

### EXAMPLE 2

In January, 2014, in Wuping Town, Jingxi County, Guangxi Province, the inventors planted 1-year-old Panax notoginseng seedlings in the soil and cultivated them according to conventional cultivation methods.

In September, 2014, pre-harvest treatment was performed. The Panax notoginseng plants were randomly divided into 6 groups: control group A, experimental group A1, experimental group A2, experimental group A3, experimental group A4 and experimental group A5, with 30 plants in each group.

In control group A, Panax notoginseng plants were sprayed with water once every 10 days, totally three times.

In the Experiment Group A1, 0.1 mmol/L salicylic acid solution was sprayed evenly on the surface of Panax notoginseng leaves, and sprayed once every 10 days, totally three times.

In the Experiment Group A2, 0.3 mmol/L salicylic acid solution was evenly sprayed on the surface of Panax notoginseng leaves, and sprayed once every 10 days, totally three times.

In the Experiment Group A3, 0.8 mmol/L salicylic acid solution was sprayed evenly on the surface of Panax notoginseng leaves, and sprayed once every 10 days, totally three times.

In the Experiment Group A4, 1.3 mmol/L salicylic acid solution was evenly sprayed on the surface of Panax notoginseng leaves, and sprayed once every 10 days, totally three times.

In the Experiment Group A5, 1.7 mmol/L salicylic acid solution was evenly sprayed on the surface of Panax notoginseng leaves, and sprayed once every 10 days, totally three times.

Salicylic acid solution preparation: 10 mmol/L salicylic acid stock solution was firstly prepared, diluted with water when further use. 10 mmol/L salicylic acid solution preparation: 1.381 g salicylic acid were weighted, dissolved in about 0.8 L of water, with 1 mol NaOH solution to adjust the pH value to 7.0, then water was added to 1 L volume.

The roots of the six treatment groups were harvested on the 10th day after the last spraying treatment. According to the method of determination of the content of Panax notoginseng in the Chinese Pharmacopoeia First Edition, notoginsenoside R1 was extracted with methanol, and the content of notoginsenoside R1 was determined by High Performance Liquid Chromatography. The experimental results of each group are shown in Table 1.

TABLE 1
the contents of notoginsenoside R1 in each treated
Panax notoginseng root

| | Number of | | |
|---|---|---|---|
| | Experimental | Treatment | Notoginsenoside R1 |
| Group | Plants | Solution | content % |
| Control A | 30 | Water | 0.23 |
| Experiment | 30 | 0.1 mmol/L | 0.25 |
| Group A1 | | Salicylic Acid | |
| | | Solution | |
| Experiment | 30 | 0.3 mmol/L | 0.28 |
| Group A2 | | Salicylic Acid | |
| | | Solution | |
| Experiment | 30 | 0.8 mmol/L | 0.38* |
| Group A3 | | Salicylic Acid | |
| | | Solution | |
| Experiment | 30 | 1.3 mmol/L | 0.36* |
| Group A4 | | Salicylic Acid | |
| | | Solution | |
| Experiment | 30 | 1.7 mmol/L | 0.27 |
| Group A5 | | Salicylic Acid | |
| | | Solution | |

*Note: n = 30, compared with Control, p < 0.05.

The experimental data in Table 1 show that the content of notoginsenoside R1 in the root of Panax notoginseng treated with salicylic acid solution increased, especially when the leaves of Panax notoginseng were treated with 0.8 mmol/L salicylic acid solution, wherein the content of notoginsenoside R1 in the root increased from 0.23% (control group A) to 0.38% (experimental group A3), increased by 65%; As can be seen from Table 1, with the concentration of salicylic acid solution increased, the content of notoginsenoside R1 was increased first and then decreased, and the optimum concentration of salicylic acid solution was 0.8 mmol/L.

### EXAMPLE 3

In January, 2013, in Wuping Town, Jingxi County, Guangxi Province, the inventors planted 1-year-old Panax notoginseng seedlings in the soil and cultivated them according to conventional cultivation methods.

In September, 2014, pre-harvest treatment was performed. The Panax notoginseng plants were randomly divided into 6 groups: control group B, experimental group B1, experimental group B2, experimental group B3, experimental group B4 and experimental group B5, with 30 plants in each group.

In control group A, Panax notoginseng plants were sprayed with water once every 10 days, totally three times.

In the Experiment Group B1, 0.1 mmol/L salicylic acid solution was sprayed evenly on the surface of Panax notoginseng leaves, and sprayed once every 10 days, totally three times.

In the Experiment Group B2, 0.3 mmol/L salicylic acid solution was evenly sprayed on the surface of Panax notoginseng leaves, and sprayed once every 10 days, totally three times.

In the Experiment Group B3, 0.8 mmol/L salicylic acid solution was sprayed evenly on the surface of Panax notoginseng leaves, and sprayed once every 10 days, totally three times.

In the Experiment Group B4, 1.3 mmol/L salicylic acid solution was evenly sprayed on the surface of Panax notoginseng leaves, and sprayed once every 10 days, totally three times.

In the Experiment Group B5, 1.7 mmol/L salicylic acid solution was evenly sprayed on the surface of Panax notoginseng leaves, and sprayed once every 10 days, totally three times.

The roots of the six treatment groups were harvested on the 10th day after the last spraying treatment. According to the method of determination of the content of Panax notoginseng in the Chinese Pharmacopoeia First Edition, notoginsenoside R1 was extracted with methanol, and the content of notoginsenoside R1 was determined by High Performance Liquid Chromatography. The experimental results of each group are shown in Table 2.

TABLE 2
the contents of notoginsenoside R1 in each treated
Panax notoginseng root

| | Number of | | |
|---|---|---|---|
| | Experimental | Treatment | Notoginsenoside |
| Group | Plants | Solution | R1 content % |
| Control B | 30 | Water | 0.39 |
| Experiment | 30 | 0.1 mmol/L Salicylic | 0.42 |
| Group B1 | | Acid Solution | |
| Experiment | 30 | 0.3 mmol/L Salicylic | 0.51 |
| Group B2 | | Acid Solution | |
| Experiment | 30 | 0.8 mmol/L Salicylic | 0.63* |
| Group B3 | | Acid Solution | |
| Experiment | 30 | 1.3 mmol/L Salicylic | 0.59* |
| Group B4 | | Acid Solution | |
| Experiment | 30 | 1.7 mmol/L Salicylic | 0.46 |
| Group B5 | | Acid Solution | |

*Note: n = 30, compared with Control, p < 0.05.

The experimental data in Table 2 show that the content of notoginsenoside R1 in the root of Panax notoginseng treated with salicylic acid solution increased, especially when the leaves of Panax notoginseng were treated with 0.8 mmol/L salicylic acid solution, wherein the content of notoginsenoside R1 in the root increased from 0.39% (control group B) to 0.63% (experimental group B3), increased by 61.5%; As can be seen from Table 2, with the concentration of salicylic acid solution increased, the content of notoginsenoside R1 was increased first and then decreased, and the optimum concentration of salicylic acid solution was 0.8 mmol/L.

### EXAMPLE 4

In January, 2012, in Wuping Town, Jingxi County, Guangxi Province, the inventors sowed the Panax notoginseng seed in January, and obtained Panax notoginseng seedlings in January, 2013, which were planted after being transplanting to obtain planting seedlings. And in mid-April the planting seedlings were cut along the circumference close to the root, and the cuts were firstly sprayed with a chitosan aqueous solution with a mass fraction of 1.35%, then coated with a plant mashing liquid, and then wrapped around with sponge with a thickness of 3-5 mm, wherein the sponge was immersed in a first soaking liquid for 20 h at a soaking temperature of 35° C. after sterilization, wherein

The chitosan in the chitosan aqueous solution was a chitosan with a N-deacetyl degree of 85%;

The plant mashing liquid is prepared by mixing aloe, moss and pepper in a mass ratio of 3:7:1, then mashing and grinding; wherein the moss was peat moss and the pepper was the pepper fruit part.

The first soaking liquid was made by mixing bamboo vinegar liquid, fatty alcohol polyoxyethylene ether sodium sulfate, peppermint essential oil and water in a mass ratio of 2:6:1:1000.

Other processes were conducted according to conventional cultivation methods.

In September, 2014, pre-harvest treatment was performed. The Panax notoginseng plants were randomly divided into 6 groups: control group C, experimental group C1, experimental group C2, experimental group C3, experimental group C4 and experimental group C5, with 30 plants in each group.

In control group C, Panax notoginseng plants were sprayed with water once every 10 days, totally three times.

In the Experiment Group C1, 0.1 mmol/L salicylic acid solution was sprayed evenly on the surface of Panax notoginseng leaves, and sprayed once every 10 days, totally three times.

In the Experiment Group C2, 0.3 mmol/L salicylic acid solution was evenly sprayed on the surface of Panax notoginseng leaves, and sprayed once every 10 days, totally three times.

In the Experiment Group C3, 0.8 mmol/L salicylic acid solution was sprayed evenly on the surface of Panax notoginseng leaves, and sprayed once every 10 days, totally three times.

In the Experiment Group C4, 1.3 mmol/L salicylic acid solution was evenly sprayed on the surface of Panax notoginseng leaves, and sprayed once every 10 days, totally three times.

In the Experiment Group C5, 1.7 mmol/L salicylic acid solution was evenly sprayed on the surface of Panax notoginseng leaves, and sprayed once every 10 days, totally three times.

The roots of the six treatment groups were harvested on the 10th day after the last spraying treatment. According to the method of determination of the content of Panax notoginseng in the Chinese Pharmacopoeia First Edition, notoginsenoside R1 was extracted with methanol, and the content of notoginsenoside R1 was determined by High Performance Liquid Chromatography. The experimental results of each group are shown in Table 3.

TABLE 3
the contents of notoginsenoside R1 in each treated
Panax notoginseng root

| | Number of | Treatment | Notoginsenoside R1 |
|---|---|---|---|
| Group | Experimental Plants | Solution | content % |
| Control C | 30 | Water | 0.45 |
| Experiment | 30 | 0.1 mmol/L | 0.49 |
| Group C1 | | Salicylic Acid | |
| | | Solution | |
| Experiment | 30 | 0.3 mmol/L | 0.61 |
| Group C2 | | Salicylic Acid | |
| | | Solution | |
| Experiment | 30 | 0.8 mmol/L | 0.77* |
| Group C3 | | Salicylic Acid | |
| | | Solution | |
| Experiment | 30 | 1.3 mmol/L | 0.73* |
| Group C4 | | Salicylic Acid | |
| | | Solution | |
| Experiment | 30 | 1.7 mmol/L | 0.53 |
| Group C5 | | Salicylic Acid | |
| | | Solution | |

*Note: n = 30, compared with Control, p < 0.05.

It can be seen from combining Table 2 and Table 3 that the planting seedlings were cut along the circumference close to the root, and the cuts were firstly sprayed with a chitosan aqueous solution, then coated with a plant mashing liquid, and then wrapped around with sponge. The content of notoginsenoside R1 increased slightly when salicylic acid was used, and the content of notoginsenoside R1 increased significantly with salicylic acid solution treatment. With the concentration of salicylic acid solution increased, the content of notoginsenoside R1 was increased first and then decreased. when the leaves of Panax notoginseng were treated with 0.8 mmol/L salicylic acid solution, the content of notoginsenoside R1 in the root increased from 0.45% (control group B) to 0.77% (experimental group B3), increased by 71.1%, while that of experimental group C3 was 22% higher than that of experimental group B3.

### EXAMPLE 5

On the basis of Example 4, in September, 2014, pre-harvest treatment was performed. The Panax notoginseng plants were randomly divided into 6 groups: control group D, experimental group D1, experimental group D2, experimental group D3, experimental group D4 and experimental group D5, with 30 plants in each group.

The salicylic acid solution was sprayed three times on the leaves of Panax notoginseng 50 days before the harvest, wherein the spraying concentration, time and method were the same as in Example 4. Non-woven sleeves were used to cover the leaves of Panax notoginseng immediately after each spray, and the non-woven sleeves were removed after 3 h, wherein the non-woven sleeves were soaked in the second soaking liquid at 30° C. for 21 h, and the second soaking liquid was prepared by mixing sodium salicylate, salicylic acid and water at a mass ratio of 4:7:1000.

The roots of the six treatment groups were harvested on the 10th day after the last spraying treatment. According to the method of determination of the content of Panax notoginseng in the Chinese Pharmacopoeia First Edition, notoginsenoside R1 was extracted with methanol, and the content of notoginsenoside R1 was determined by High Performance Liquid Chromatography. The experimental results of each group are shown in Table 4.

TABLE 4
the contents of notoginsenoside R1 in each treated
Panax notoginseng root

| | Number of | | |
|---|---|---|---|
| | Experimental | Treatment | Notoginsenoside R1 |
| Group | Plants | Solution | content % |
| Control D | 30 | Water | 0.48 |
| Experiment | 30 | 0.1 mmol/L | 0.53 |
| Group D1 | | Salicylic Acid | |
| | | Solution | |
| Experiment | 30 | 0.3 mmol/L | 0.66 |
| Group D2 | | Salicylic Acid | |
| | | Solution | |
| Experiment | 30 | 0.8 mmol/L | 0.89* |
| Group D3 | | Salicylic Acid | |
| | | Solution | |
| Experiment | 30 | 1.3 mmol/L | 0.84* |
| Group D4 | | Salicylic Acid | |
| | | Solution | |
| Experiment | 30 | 1.7 mmol/L | 0.61 |
| Group D5 | | Salicylic Acid | |
| | | Solution | |

*Note: n = 30, compared with Control, p < 0.05.

It can be seen from combining Table 3 and Table 4 that after treatment with non-woven sleeves immersed with the second soaking liquid, the content of notoginsenoside R1 were slightly increased, and increase rate of the experimental group D3 was more obvious, among them, compared with the experimental group C3, the notoginsenoside R1 content of experimental group D3 increased by 15.58%, and the increase rates of other experimental groups were relatively low.

### EXAMPLE 6

On the basis of Example 4, in March, 2014, a plurality of fine pores were punched along the length direction of the stem in the trunk of the Panax notoginseng plants, and a sulfonic acid aqueous solution was sprayed on the fine pores using a sprayer, wherein the aqueous solution were prepared by mixing sulfonic acid, sodium cocoyl isethionate and water in a mass ratio of 7:4:10000.

In September, 2014, pre-harvest treatment was performed as described in Example 4. he Panax notoginseng plants were randomly divided into 6 groups: control group E, experimental group E1, experimental group E2, experimental group E3, experimental group E4 and experimental group E5, with 30 plants in each group.

The roots of the six treatment groups were harvested on the 10th day after the last spraying treatment. According to the method of determination of the content of Panax notoginseng in the Chinese Pharmacopoeia First Edition, notoginsenoside R1 was extracted with methanol, and the content of notoginsenoside R1 was determined by High Performance Liquid Chromatography. The experimental results of each group are shown in Table 5.

TABLE 5
the contents of notoginsenoside R1 in each treated
Panax notoginseng root

| | Number of | | |
|---|---|---|---|
| | Experimental | Treatment | Notoginsenoside R1 |
| Group | Plants | Solution | content % |
| Control E | 30 | Water | 0.46 |
| Experiment | 30 | 0.1 mmol/L | 0.55 |
| Group E1 | | Salicylic Acid | |
| | | Solution | |
| Experiment | 30 | 0.3 mmol/L | 0.61 |
| Group E2 | | Salicylic Acid | |
| | | Solution | |
| Experiment | 30 | 0.8 mmol/L | 0.83* |
| Group E3 | | Salicylic Acid | |
| | | Solution | |
| Experiment | 30 | 1.3 mmol/L | 0.81* |
| Group E4 | | Salicylic Acid | |
| | | Solution | |
| Experiment | 30 | 1.7 mmol/L | 0.59 |
| Group E5 | | Salicylic Acid | |
| | | Solution | |

*Note: n = 30, compared with Control, p < 0.05.

It can be seen from combining Table 3 and Table 5 that in March, 2014, a plurality of fine pores were punched along the length direction of the stem in the trunk of the Panax notoginseng plants, and a sulfonic acid aqueous solution was sprayed on the fine pores using a sprayer, the content of notoginsenoside R1 increased, and the increase rate in the experimental group E3 was more obvious, wherein compared with the experimental group C3, the notoginsenoside R1 content of experimental group E3 increased by 7.8%.

### EXAMPLE 6

On the basis of Example 4, before the sowing, the Panax notoginseng seeds were sterilized, then 3-5 holes were pinned on the surface of the Panax notoginseng seed. The seeds were further immersed in a third soaking liquid at 34° C. for 6 h, coated with a film for 4 minutes and then removing the film, wherein the third soaking liquid was prepared by mixing salicylic acid, water-soluble chitosan, lanolin, ethanol and water in a mass ratio of 1:8:5:200:800.

In September, 2014, pre-harvest treatment was performed as described in Example 4. he Panax notoginseng plants were randomly divided into 6 groups: control group F, experimental group F1, experimental group F2, experimental group F3, experimental group F4 and experimental group F5, with 30 plants in each group.

The roots of the six treatment groups were harvested on the 10th day after the last spraying treatment. According to the method of determination of the content of Panax notoginseng in the Chinese Pharmacopoeia First Edition, notoginsenoside R1 was extracted with methanol, and the content of notoginsenoside R1 was determined by High Performance Liquid Chromatography. The experimental results of each group are shown in Table 6.

TABLE 6
the contents of notoginsenoside R1 in each treate
Panax notoginseng root

| | Number of | | |
|---|---|---|---|
| | Experimental | Treatment | Notoginsenoside R1 |
| Group | Plants | Solution | content % |
| Control F | 30 | Water | 0.46 |
| Experiment | 30 | 0.1 mmol/L | 0.50 |
| Group F1 | | Salicylic Acid | |
| | | Solution | |
| Experiment | 30 | 0.3 mmol/L | 0.64 |
| Group F2 | | Salicylic Acid | |
| | | Solution | |
| Experiment | 30 | 0.8 mmol/L | 0.84* |
| Group F3 | | Salicylic Acid | |
| | | Solution | |
| Experiment | 30 | 1.3 mmol/L | 0.76* |
| Group F4 | | Salicylic Acid | |
| | | Solution | |
| Experiment | 30 | 1.7 mmol/L | 0.59 |
| Group F5 | | Salicylic Acid | |
| | | Solution | |

*Note: n = 30, compared with Control, p < 0.05.

It can be seen from combining Table 3 and Table 6 that before the sowing, 3-5 holes were pinned on the surface of the Panax notoginseng seed. The seeds were further immersed in a third soaking liquid, coated with a film and then removing the film, the content of notoginsenoside R1 increased to a great extent, wherein compared with the experimental group C3, the notoginsenoside R1 content of experimental group F3 increased by about 9%.

Although the embodiments of the present invention have been disclosed above, the present invention is not limited to the applications set forth in the specification and the embodiments, and it can be applied to various fields suitable for the present invention, and it is easy for those skilled in the art to make additional modifications. The present invention is not limited to the specific details and embodiments shown and described herein, without departing from the general concept defined in the claims and the equivalents.

What is claimed is:

1. A method for increasing the content of notoginsenoside R1 in Panax notoginseng, characterized in that Panax notoginseng plants are sprayed with salicylic acid solution before the Panax notoginseng harvest.

2. The method for improving the content of Notoginsenoside R1 in Panax notoginseng according to claim 1, characterized in that the salicylic acid solution has a concentration of 0.3-1.3 mmol/L.

3. The method for improving the content of Notoginsenoside R1 in Panax notoginseng according to claim 2, characterized in that the Panax notoginseng plants are sprayed with the salicylic acid solution on 30 to 70 days before the Panax notoginseng harvest.

4. The method for improving the content of Notoginsenoside R1 in Panax notoginseng according to claim 3, characterized in that the Panax notoginseng plants are those which have been cultivated for 2 to 3 years.

5. The method for improving the content of Notoginsenoside R1 in Panax notoginseng according to claim 4, characterized in that the salicylic acid solution is sprayed on the leaves surface of the Panax notoginseng plants.

6. The method for improving the content of Notoginsenoside R1 in Panax notoginseng according to claim 2, characterized in that the Panax notoginseng plants are sprayed with the salicylic acid solution once every 10-20 days before harvest, totally 2-3 times.

7. The method for improving the content of Notoginsenoside R1 in Panax notoginseng according to claim 6, characterized in that Panax notoginseng seeds are sowed in January of the first year, and Panax notoginseng seedlings are obtained in January of the second year, which are planted after being transplanting to obtain planting seedlings, and in April to May the planting seedlings are cut along the circumference close to the root, and the cuts are firstly sprayed with a chitosan aqueous solution with a mass fraction of 1.2%-1.5%, then coated with a plant mashing liquid, and then wrapped around with sponge with a thickness of 3-5 mm, wherein the sponge is immersed in the first soaking liquid for 20-22 h at a soaking temperature of 33-35° C. after sterilization, wherein
the chitosan in the chitosan aqueous solution is a chitosan with a N-deacetyl degree of 85% -88%;
the plant mashing liquid is prepared by mixing aloe, moss and pepper in a mass ratio of 3:7:1, then mashing and grinding;
the first soaking liquid is made by mixing bamboo vinegar liquid, fatty alcohol polyoxyethylene ether sodium sulfate, peppermint essential oil and water in a mass ratio of 2-3:5-7:1-2:1000.

8. The method for improving the content of Notoginsenoside R1 in Panax notoginseng according to claim 7, characterized in that 30 days before the Panax notoginseng harvest, the salicylic acid solution is sprayed on the leaves of the Panax notoginseng plant at an interval of 10 days, totally 3 times, and non-woven sleeves are used for covering leaves of Panax notoginseng plants, then removed after 2-4 h, wherein the non-woven sleeves are immersed in the second soaking solution at 28-30° C. for 20-22 h, and the second soaking liquor is prepared by mixing sodium salicylate, salicylic acid and water at a mass ratio of 4-5:7-8:1000.

9. The method for improving the content of Notoginsenoside R1 in Panax notoginseng according to claim 7, characterized in that in March of the third year, a plurality of pores is drilled on the main stem of the Panax notoginseng plant along the length direction of the main stem, and then sulfonic acid aqueous solution is sprayed with a sprayer on the pores, wherein the sulfonic acid aqueous solution is prepared by mixing sulfonic acid, sodium cocoyl isethionate and water in a mass ratio of 7:3-5:10000.

10. The method for improving the content of Notoginsenoside R1 in Panax notoginseng according to claim 7, characterized in that before the sowing, the Panax notoginseng seeds are sterilized, then 3-5 holes are pinned on the surface of the Panax notoginseng seed, and the seeds are further immersed in a third soaking liquid at 35° C. for 5-7 hours, coated with a film for 3-5 minutes and then removing the film, wherein the third soaking liquid is prepared by mixing salicylic acid, water-soluble chitosan, lanolin, ethanol and water in a mass ratio of 1:7-9:5:200:800.
